(19) United States
(12) Patent Application Publication
(10) Pub. No.: US 2024/0212648 A1
(43) Pub. Date: Jun. 27, 2024

(54) OUTPUTTING REPRESENTATIONS HAVING LEVELS OF LIGHT ILLUMINATION THAT ARE BASED ON A USER'S RATE OF EYE ADAPTATION

(71) Applicant: International Business Machines Corporation, Armonk, NY (US)

(72) Inventors: Sarbajit K. Rakshit, Kolkata (IN); Shailendra Moyal, Pune (IN)

(21) Appl. No.: 18/087,654

(22) Filed: Dec. 22, 2022

(51) Int. Cl. G09G 5/10; G06T 19/00; G06T 19/20

(52) U.S. Cl. CPC G09G 5/10; G06T 19/006; G06T 19/20; A61B 3/063

(57) ABSTRACT

A computer-implemented method, according to one embodiment, includes learning a rate of eye adaptation that a first user's pupil muscle adjusts. In response to a determination that the first user is moving from a first location having a first level of light illumination to a second location having a second level of light illumination, it is determined whether a difference in the levels has a potential for causing eye adaptation issues for the first user. In response to a determination that the difference in the levels has a potential for causing eye adaptation issues, a level of light illumination that will allow the first user's pupil muscle to adjust at the learned rate of eye adaptation is determined. A first representation of the second location is output for display on a mixed reality device worn by the first user upon the first user entering the second location.

### BACKGROUND

The present invention relates to eye adaptation to light, and more specifically, this invention relates to outputting representations, for display on a mixed reality (MR) device, having levels of light illumination that are based on a user's rate of eye adaptation.

In visual physiology, adaptation is the ability of the retina of the eye to adjust to various levels of light. Natural night vision, or scotopic vision, is the ability to see under low-light conditions. In humans, rod cells are exclusively responsible for night vision as cone cells are only able to function at higher illumination levels. Night vision is of relatively lower quality than day vision because it is limited in resolution and colors cannot be discerned; only shades of gray are seen. In order for humans to transition from day to night vision they must undergo a dark adaptation period of up to two hours during which each eye adjusts from a high to a low luminescence “setting”, increasing sensitivity hugely, by many orders of magnitude. This adaptation period is different between rod and cone cells and results from the regeneration of photopigments to increase retinal sensitivity. Light adaptation, in contrast, occurs relatively quickly, e.g., within seconds.

### SUMMARY

A computer-implemented method, according to one embodiment, includes learning a rate of eye adaptation that a first user's pupil muscle adjusts to a change in a perceived level of light illumination. In response to a determination that the first user is moving from a first location having a first level of light illumination to a second location having a second level of light illumination, it is determined whether a difference in the levels of light illumination has a potential for causing eye adaptation issues for the first user. In response to a determination that the difference in the levels of light illumination has a potential for causing eye adaptation issues for the first user, a level of light illumination that will allow the first user's pupil muscle to adjust at the learned rate of eye adaptation is determined. The method further includes outputting a first representation of the second location for display on a mixed reality (MR) device worn by the first user upon the first user entering the second location. The first representation of the second location includes the determined level of light illumination.

A computer program product, according to another embodiment, includes a computer readable storage medium having program instructions embodied therewith. The program instructions are readable and/or executable by a computer to cause the computer to perform the foregoing method.

A system, according to another embodiment, includes a processor, and logic integrated with the processor, executable by the processor, or integrated with and executable by the processor. The logic is configured to perform the foregoing method.

Other aspects and embodiments of the present invention will become apparent from the following detailed description, which, when taken in conjunction with the drawings, illustrate by way of example the principles of the invention.

### BRIEF DESCRIPTION OF THE DRAWINGS

FIG. 1 is a diagram of a computing environment, in accordance with one embodiment of the present invention.

FIG. 2A is a flowchart of a method, in accordance with one embodiment of the present invention.

FIG. 2B is a flowchart of sub-operations of an operation of the flowchart of FIG. 2A, in accordance with one embodiment of the present invention.

FIG. 3A is a perspective of a first location, in accordance with one embodiment of the present invention.

FIG. 3B is a perspective of a second location, in accordance with one embodiment of the present invention.

FIG. 3C is a representation of the perspective of the second location of FIG. 3B displayed on a mixed reality (MR) device, in accordance with one embodiment of the present invention.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The following description discloses several preferred embodiments of systems, methods and computer program products for outputting representations, for display on a mixed reality (MR) device, having levels of light illumination that are based on a user's rate of eye adaptation.

In one general embodiment, a computer-implemented method includes learning a rate of eye adaptation that a first user's pupil muscle adjusts to a change in a perceived level of light illumination. In response to a determination that the first user is moving from a first location having a first level of light illumination to a second location having a second level of light illumination, it is determined whether a difference in the levels of light illumination has a potential for causing eye adaptation issues for the first user. In response to a determination that the difference in the levels of light illumination has a potential for causing eye adaptation issues for the first user, a level of light illumination that will allow the first user's pupil muscle to adjust at the learned rate of eye adaptation is determined. The method further includes outputting a first representation of the second location for display on a mixed reality (MR) device worn by the first user upon the first user entering the second location. The first representation of the second location includes the determined level of light illumination.

In another general embodiment, a computer program product includes a computer readable storage medium having program instructions embodied therewith. The program instructions are readable and/or executable by a computer to cause the computer to perform the foregoing method.

In another general embodiment, a system includes a processor, and logic integrated with the processor, executable by the processor, or integrated with and executable by the processor. The logic is configured to perform the foregoing method.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as light illumination level determination module of block 200 for determining representations having levels of light illumination that are based on a user's rate of eye adaptation. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IOT) sensor set 125), and network module 115. Remote server 104 includes remote database 130. Public cloud 105 includes gateway 140, cloud orchestration module 141, host physical machine set 142, virtual machine set 143, and container set 144.

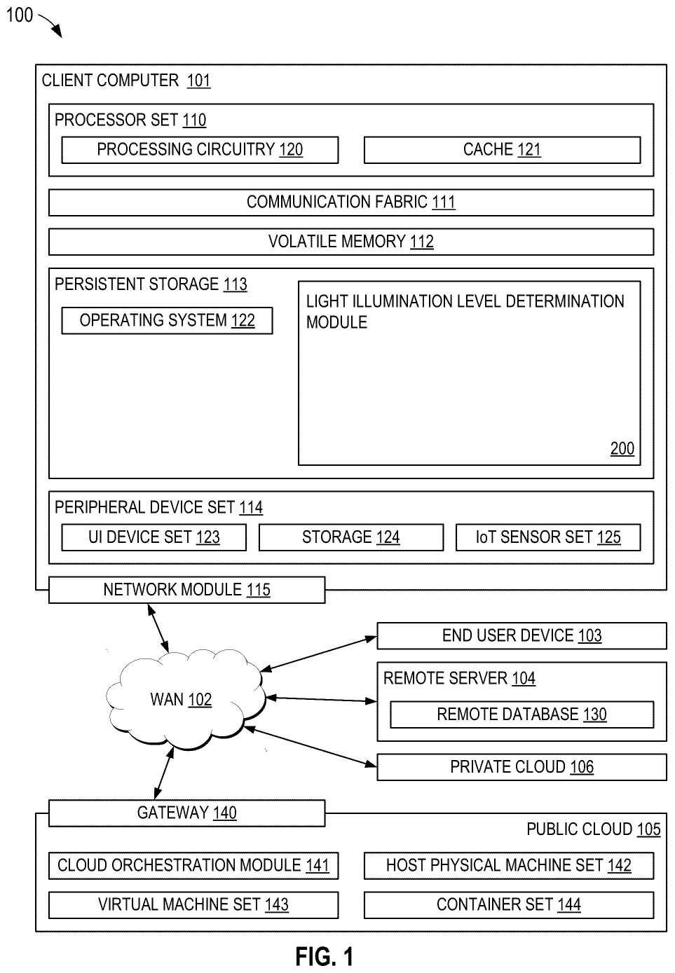

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

In some aspects, a system according to various embodiments may include a processor and logic integrated with and/or executable by the processor, the logic being configured to perform one or more of the process steps recited herein. The processor may be of any configuration as described herein, such as a discrete processor or a processing circuit that includes many components such as processing hardware, memory, I/O interfaces, etc. By integrated with, what is meant is that the processor has logic embedded therewith as hardware logic, such as an application specific integrated circuit (ASIC), a FPGA, etc. By executable by the processor, what is meant is that the logic is hardware logic; software logic such as firmware, part of an operating system, part of an application program; etc., or some combination of hardware and software logic that is accessible by the processor and configured to cause the processor to perform some functionality upon execution by the processor. Software logic may be stored on local and/or remote memory of any memory type, as known in the art. Any processor known in the art may be used, such as a software processor module and/or a hardware processor such as an ASIC, a FPGA, a central processing unit (CPU), an integrated circuit (IC), a graphics processing unit (GPU), etc.

Of course, this logic may be implemented as a method on any device and/or system or as a computer program product, according to various embodiments.

As mentioned elsewhere herein, in visual physiology, adaptation is the ability of the retina of the eye to adjust to various levels of light. Natural night vision, or scotopic vision, is the ability to see under low-light conditions. In humans, rod cells are exclusively responsible for night vision as cone cells are only able to function at higher illumination levels. Night vision is of relatively lower quality than day vision because it is limited in resolution and colors cannot be discerned; only shades of gray are seen. In order for humans to transition from day to night vision they must undergo a dark adaptation period of up to two hours during which each eye adjusts from a high to a low luminescence “setting”, increasing sensitivity hugely, by many orders of magnitude. This adaptation period is different between rod and cone cells and results from the regeneration of photopigments to increase retinal sensitivity. Light adaptation, in contrast, occurs relatively quickly, e.g., within seconds.

On any industrial floor, e.g., such as an industrial floor of a manufacturing plant, workers may move from one location to another location of the industrial floor in a normal course of work duties. Based on this mobility, a worker may move from an area having a first level of light illumination, e.g., relatively lighter, to an area having a second level of light illumination, e.g., relatively darker, and vice versa. For a relatively short period of time, the workers may experience difficulty visualizing their surroundings. This may cause an accident, as the workers are not able to visualize their surroundings. This problem is particularly apparent in settings that have an area dedicated for activities that are to be performed in the dark, e.g., such as an area where photosensitive chemicals are stored. Accordingly, there is a need to help users with eye adaptation issues experienced as a result of entering an area that has a different level of light illumination than a level of light illumination of a most previous location of the user.

In sharp contrast to the deficiencies described above, techniques of various embodiments and approaches described herein include learning a rate of eye adaptation that a user's pupil muscle adjusts to a change in a perceived level of light illumination. Furthermore, a level of light illumination that will allow the user's pupil muscle to adjust at the learned rate of eye adaptation is included in representations output to display on a mixed reality (MR) device worn by the user. This way, the user is safely able to continue working while at the same time the user's pupil muscle is able to gradually adjust to the changing levels of light illumination.

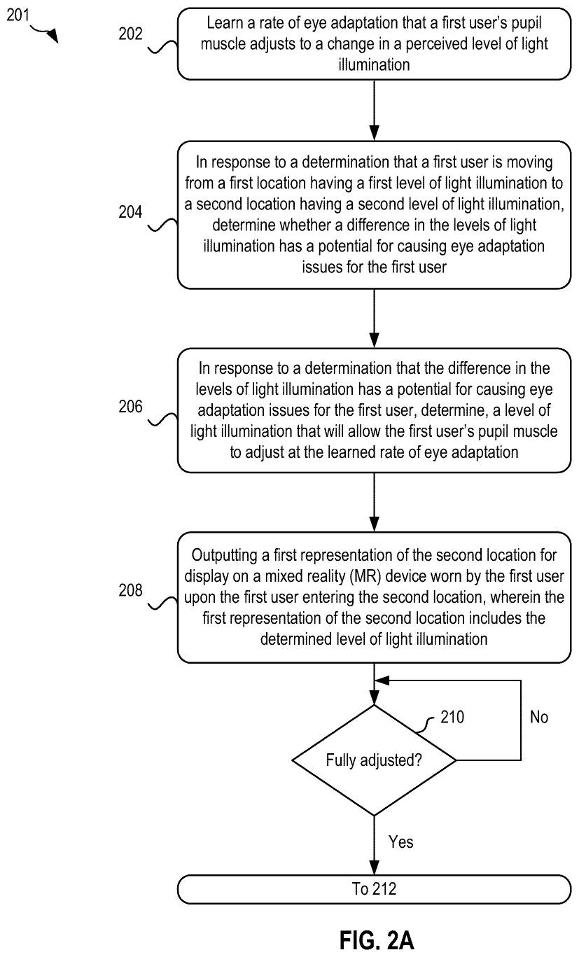

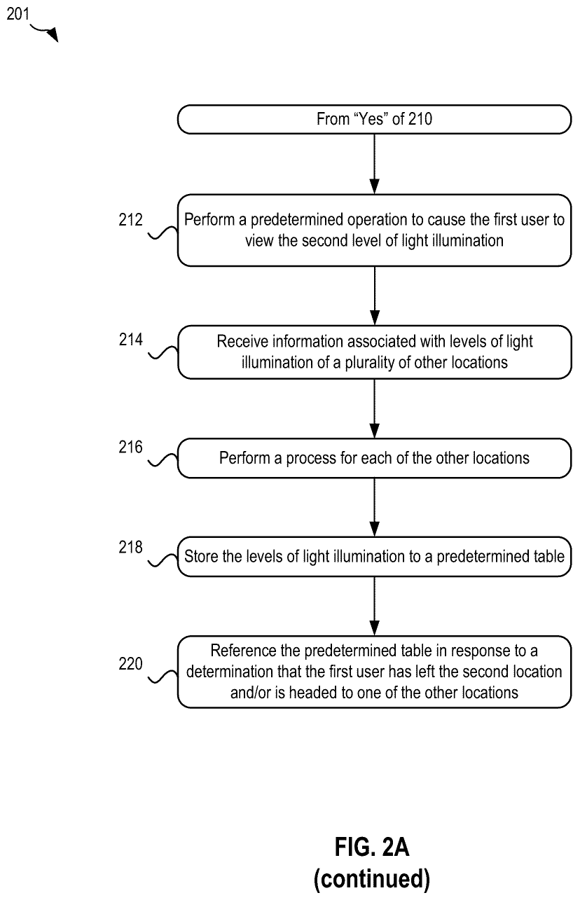

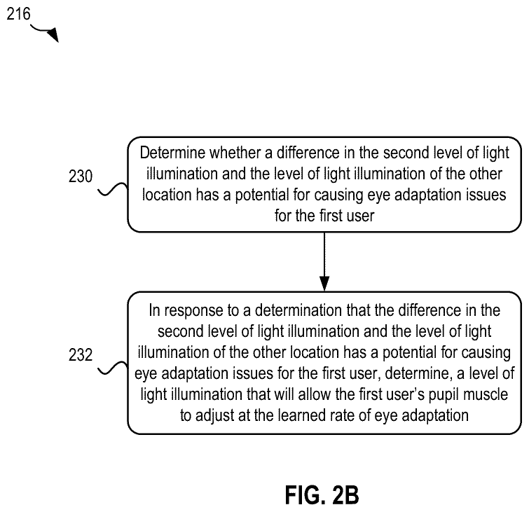

Now referring to FIG. 2A, a flowchart of a method 201 is shown, according to one embodiment. The method 201 may be performed in accordance with the present invention in any of the environments depicted in FIGS. 1-3C, among others, in various embodiments. Of course, more or fewer operations than those specifically described in FIG. 2 may be included in method 201, as would be understood by one of skill in the art upon reading the present descriptions.

Each of the steps of the method 201 may be performed by any suitable component of the operating environment. For example, in various embodiments, the method 201 may be partially or entirely performed by a computer, or some other device having one or more processors therein. The processor, e.g., processing circuit(s), chip(s), and/or module(s) implemented in hardware and/or software, and preferably having at least one hardware component may be utilized in any device to perform one or more steps of the method 201. Illustrative processors include, but are not limited to, a central processing unit (CPU), an application specific integrated circuit (ASIC), a field programmable gate array (FPGA), etc., combinations thereof, or any other suitable computing device known in the art.

For context, it may be prefaced that on any industrial floor, different portions of the surroundings will have different levels of illumination, e.g., light illumination. For example, in some approaches, different locations of the industrial floor may be maintained to have different levels of light illumination for one reason or another. In some approaches, the relative light illumination of one or more of these locations may be relatively darkened by, e.g., enclosing a room, turning off lights, it being nighttime, curtains, etc. In some other approaches, the relative light illumination of one or more of these locations may be relatively lightened by, e.g., opening a door, opening blinds and/or curtains, turning on lights, it being daytime, windows letting in light, etc.

Eye adaptation may be defined as a time required for a user to adapt to a current lighting condition. In relatively bright light the user's pupil size will be small, which thereby only allows a small portion of light to enter the eye. This pupil size will not be visible in a relatively dark surrounding because the user's pupil muscle opens up the size of the pupil to be relatively larger. Note that different people may require different times for eye adaptation. Operation 202 includes learning a rate of eye adaptation that a first user's pupil muscle adjusts to a change in a perceived level of light illumination. For context, the level of light illumination is “perceived” based on the light illumination being taken in by a retina of the first user's eye. A known type of pupil dilation monitoring device may be used to learn the first user's rate of eye adaptation. In some other approaches, method 201 may include identifying a muscle flexion of the pupil of the first user, e.g., using a pupil tracking camera. The muscle flexion may, in some approaches, be considered during the learning of the rate of eye adaptation that the first user's pupil muscle adjusts. In some other approaches, one or more techniques that would become apparent to one of ordinary skill in the art may be additionally and/or alternatively used for learning the rate of eye adaptation that the first user's pupil muscle adjusts to the change in the perceived level of light illumination.

In some approaches, a series of trial operations may be performed in which the first user's eye is subjected to a predetermined number of different light illuminations. In some other approaches, the first user may wear a known type of MR device and/or virtual reality device, and the first user's eye(s) may be monitored for a predetermined period of time as the first user performs normal daily tasks. For example, in some approaches, based on one or more usage patterns of the users, the MR device may be configured to collect information that is processed to learn learning rates of eye adaptation of different users with respect to entering into different level of lighting.

In some other approaches, the learning may additionally and/or alternatively be based on a factor such as historical learning about the level of illumination of the user. In another approach, the learning may be based on a comfort level of one or more workers to have required level of illumination. This amount of comfort may be set in response to receiving user input. In some other approaches, the learning may be based on time required and/or observed for eye adaptation because different users may have different timing for such adaptation. Accordingly, the MR device may be used to learn adaptation rates in a personalized manner for a plurality of users. In one preferred approach, the MR device worn by the user includes a display which may be configured to be, e.g., transparent when not displaying a representation which thereby allows a user wearing the MR device to see through the display, semi-transparent when displaying a representation which thereby allows a user wearing the MR device to see through the display and also see one or more portions of the representation displayed on the screen, not transparent at all which thereby only allows the user to view the representation when displayed on the screen. In some approaches, while working throughout an industrial floor, users may wear AR and/or MR glass for eye protection, and as will be described below, in order to receive a predetermined amount of eye adaptation support.

With a user's rate of eye adaptation learned, the user may be monitored to determine whether the user is moving among locations with different respective levels of light illumination. One or more types of sensors may be present at the first location and/or the second location and/or the industrial floor to be used for such monitoring and/or to gather parameters of illumination. It should be prefaced that, monitoring of users is preferably only performed subsequent to gaining permission by the users to do so, e.g., an opt-in parameter. During such monitoring, a location of the user and/or a direction of mobility of the user may be monitored to determine whether the user is moving from a first location having a first level of light illumination to a second location having a second level of light illumination. For example, the first level of light illumination may be relatively darker, and the second level of light illumination may be relatively lighter. In contrast, the first level of light illumination may be relatively lighter, and the second level of light illumination may be relatively darker. In some other approaches, the first level of light illumination may be about the same as the second level of light illumination. However, such an approach is unlikely to cause eye adaptation issues for the first user, and therefore may optionally not call for a supplemental level of light illumination that will allow the first user's pupil muscle to adjust at the learned rate of eye adaptation.

In response to a determination that a first user is moving from a first location having a first level of light illumination to a second location having a second level of light illumination, it is determined whether a difference in the levels of light illumination has a potential for causing eye adaptation issues for the first user, e.g., see operation 204. Accordingly, method 201 may include determining changes in levels of light illumination in locations that the user is at and/or directionally headed towards. In some approaches, this location information may be determined, e.g., based on a work schedule of the user, from received user input, from a work scheduler, etc., which may indicate activities that the first user is to perform in the second location and/or a surrounding. For example, in some approaches, the difference may be compared with a predetermined threshold that is determined based on the learned rate of eye adaption for the first user. In another approach, it may be determined, based on the difference and the first user's learned rate of eye adaption whether the user will be able to make the adjustment within a predetermined amount of time. It may be determined that the difference in the levels of light illumination has a potential for causing eye adaptation issues for the first user in response to a determination that the user will be able to make the adjustment within a predetermined amount of time. Such a predetermined amount of time may, in some approaches, be set based on an amount of time that the user should be ready to avoid an obstacle within the second predetermined location upon entering into the second location.

In some approaches, at least one of the locations is indoors, e.g., such as in an industrial building or a manufacturing facility with a plurality of rooms. In one or more of such approaches, the level of light illumination of the indoor location may be output by a light emitting device, e.g., lightbulbs, light emitting diodes (LEDs), etc. The level of light illumination of the indoor location may additionally and/or alternatively be output by, e.g., sunlight, moonlight, ultraviolet light, etc.

In some other approaches, based on a level of difference in the brightness between the first location and the second location, historical learning may be used to identify an expected pupil size change. Accordingly, a determination of whether the levels of light illumination have a potential for causing eye adaptation issues for the first user may additionally and/or alternatively be based on the expected pupil size change. For example, in response to a determination that the expected pupil size change is greater than a predetermined threshold, it may be determined that there is a potential for causing eye adaptation issues. In contrast, in response to a determination that the expected pupil size change is less than a predetermined threshold, it may be determined that there is not a potential for causing eye adaptation issues.

In response to a determination that the difference in the levels of light illumination has a potential for causing eye adaptation issues for the first user, a level of light illumination that will allow the first user's pupil muscle to adjust at the learned rate of eye adaptation is determined, e.g., see operation 206. In other words, an appropriate level of illumination of a surrounding of the second location may proactively be created in the MR device display, which may have a deviation from the level of light illumination of the second setting. In some approaches, the level of illumination may be based on worker specific information. For example, in one or more of such approaches, the level of illumination may be an identified level of brightness that is called for in order in a surrounding of the second location for the user to perform one or more predetermined work activities, e.g., a minimum threshold of light.

In some approaches, determined level of light illumination may be created by using one or more predetermined MR capabilities. For example, a first representation of at least the second location may be output for display on a MR device worn by the first user upon the first user entering the second location, e.g., see operation 208. In such an example, the first representation of the second location includes the determined level of light illumination. This way, the first user's eyes may be adjusted without having to apply or dim light in the actual surrounding of the second location, and the user can clearly visualize the surrounding with the MR system. This ultimately increases relative productivity of a location, e.g., an industrial floor, which includes the second location for at least two reasons. First, the first user is able to enter the second location, and as a result of viewing the first representation of at least the second location may be output for display on a MR device, the first user does not experience eye adaptation issues. Second, other users at the second location, whose eyes are likely already adjusted to the level of light illumination to a second location, are not interrupted by the lights otherwise being dimmed or increased to accommodate the eye adaptation of the first user. Accordingly, in some approaches, method 201 includes not adjusting the levels of light illumination in the first location and the second location, e.g., light emitted from fixtures at such locations. Instead, the levels of light illumination remain the same to allow the first user's pupil muscle to adjust at the learned rate of eye adaptation.

The first representation may include at least some of the contents of the second location. For example, one or more predetermined target items that the first user may come across or use while at the second location may be displayed with the determined level of light illumination in the display of the MR device. One or more objects outside of the second location may also be included in the first representation, e.g., thereby allowing the user to look outside of the second location as if the user is in a virtual environment. In some approaches, an IoT feed may be received. Method 201 may include, using the IoT feed to identify objects that are in a surrounding of the first user at the second location and determining which of such objects to have the determined level of light illumination. In one preferred approach, the determined level of light illumination may be included on the target object in the first representation, so that the first user can view their physical surroundings, e.g., using the MR device and/or an AR device. In some approaches, method 201 may include communicating with IoT ecosystem in a surrounding of the second location to provide context of user's activity after moving from dark to light or light to dark etc.

The level of light illumination may, in some approaches, be less than the level of light illumination of the second location. For example, a predetermined fraction of the level of light illumination of the second location may be set as the level of light illumination of the first representation of the second location. This lesser level of light illumination may allow the first user to relatively quickly adjust to the difference in the levels of light illumination between the first location and the second location. More specifically, the first user does not experience a “white out” flash that would otherwise potentially hurt the user's eyes and potentially cause a headache for the first user in accordance with experiencing the eye adaptation issues.

In some approaches, a series of updates may be performed to the level of light illumination of the first representation in order to gradually ramp up the level of light illumination of the first representation as the first user's pupil muscle uses the first representation to adjust to the level of light illumination at the second location. For example, method 201 optionally includes updating, at a first predetermined rate, the level of light illumination of the first representation, e.g., increasing or decreasing the light to help the first user's pupil muscle to seamlessly continue to adjust at the learned rate of eye adaptation. The first predetermined rate may, in some approaches, about match the learned rate of eye adaptation that the first user's pupil muscle adjusts to a change in a perceived level of light illumination, e.g., see operation 202.

It may be determined, subsequent to the outputting of the first representation, whether the first user's pupil muscle is adjusting at the learned rate of eye adaptation. One or more techniques that were used to learn the rate of eye adaptation of the first user's pupil muscle may be used to determine whether the first user's pupil muscle is adjusting at the learned rate of eye adaptation as a result of looking at the first representation in the MR device. In response to a determination that the first user's pupil muscle is adjusting at about, e.g., within a predetermined percentage (1%, 5%, 10%, etc.) of the variance, the learned rate of eye adaptation, the level of light illumination of the first representation may continue to be updated at the first predetermined rate. In contrast, in response to a determination that the first user's pupil muscle is not adjusting at about, e.g., within the predetermined percentage of the variance, the learned rate of eye adaptation, the level of light illumination of the first representation may be updated at a second predetermined rate. The second predetermined rate may adjust the first predetermined rate a predetermined amount in an attempt to cause the first user's pupil muscle to adjust at about the learned rate of eye adaptation.

In some approaches, method 201 may include determining and monitoring a health of the first user's eye(s) to ensure that the first user is not straining their eye(s). For example, it may be determined whether the rate of eye adaptation of the user has gradually decreased, e.g., an average rate of eye adaptation for a first predetermined period of time is less than a previous average rate of eye adaptation for a second predetermined period of time.

As the method continues, eventually the first user's pupil muscle may become fully adjusted to the second level of light illumination at the second location, e.g., see decision 210. This determination may, in some approaches, be made in response to an estimated amount of adjustment time passing, e.g., an amount of time that it takes for the first user's eyes to adjust to the second location. Accordingly, it may be determined whether the first user's pupil muscle has fully adjusted to the second level of light illumination at the second location, e.g., see decision, 201. In some approaches, this determination may be based on, e.g., the first user's pupil muscle no longer adjusting thereby indicating that a full adjustment has been made, the first user's pupil muscle reaching a predetermined size that is associated with the level of light illumination of the second location, etc.

In response to a determination that the first user's pupil muscle has not become fully adjusted to the second level of light illumination at the second location, e.g., as illustrated by the “No” logical path of decision 210, the determination may be made again, e.g., subsequent to a predetermined amount of time passing. In contrast, in response to a determination that the first user's pupil muscle has fully adjusted to the second level of light illumination, e.g., as illustrated by the “Yes” logical path of decision 210, a predetermined operation may be performed to cause the first user to view the second level of light illumination, e.g., see operation 212. In other words, the first user no longer has a need to view an altered state of brightness of the second location. In one approach, the predetermined operation may include not outputting anything to be displayed on the MR device worn by the first user, e.g., thereby allowing the user to look through a transparent screen of the MR device. In another approach, the predetermined operation may include physically adjusting, e.g., flipping up, the display of the MR device so that the first user sees the second location without any portion of the MR device obstructing the first user's view. In yet another approach, the predetermined operation may include outputting a second representation of at least the second location for display on the MR device, where the second representation of the second location includes the second level of light illumination. Even after the first user's eye(s) fully adjust, monitoring may continue to be performed to ensure that the first user's pupil size is being adapted and/or remains adapted to the new environment. In some approaches, in response to a determination that the first user's pupil is no longer fully adjusted, further representations may be output to gradually bring the user's eye to complete normal eye viewing, so that MR device can optimize power consumption.

Operation 214 includes obtaining, e.g., receiving, requesting, measuring, querying, etc., information associated with levels of light illumination of a plurality of other locations. Such information may be obtained in order to, ahead of time, determine relative differences in light among different locations. This way, in the event that the first user leaves the second location, e.g., such as to a third location, a difference of relative levels of light illumination may be readily determined. Operation 216 includes performing a process for each of the other locations. Looking to FIG. 2B, exemplary sub-operations for performing a process for each of the other locations are illustrated in accordance with one embodiment, one or more of which may be used to perform operation 216 of FIG. 2A. However, it should be noted that the sub-operations of FIG. 2B are illustrated in accordance with one embodiment which is in no way intended to limit the invention. Sub-operation 230 includes determining whether a difference in the second level of light illumination and the level of light illumination of the other location has a potential for causing eye adaptation issues for the first user. Sub-operation 232 includes, in response to a determination that the difference in the second level of light illumination and the level of light illumination of the other location has a potential for causing eye adaptation issues for the first user, determining, a level of light illumination that will allow the first user's pupil muscle to adjust at the learned rate of eye adaptation. With reference again to FIG. 2A, the levels of light illumination, e.g., determined in sub-operation 232 may be stored to a predetermined table, e.g., see operation 218. Such a predetermined table may be referenced in response to a determination that the first user has left the second location and/or is headed to one of the other locations, e.g., see operation 220.

The representations output for display on the MR device may, in some approaches, include adjustments to color of one or more of the locations. For example, in one approach, method 201 optionally includes adjusting a color of one or more articles of clothing of a second user in the first representation of at least the second location. The adjusted color of the one or more articles of clothing may, in some approaches, be based on and thereby indicate a job task of the second user. For example, workers having predetermined work responsibilities may be set to have the same color. For example, based on historical learning, it may be determined that uniforms of one or more users at the second location is red. Accordingly, in response to this color already being used for a uniform color, the color yellow may be used to visualize other users clearly and/or a path of the second location. This improves work efficiency, and potentially safety of the second location in the event that colors of clothing are relatively brightened to make users relatively more noticeable in the displayed representation of the MR device display.

A color of a contour of a floor of one or more locations may additionally and/or alternatively be adjusted. For example, a color of a contour of a floor of the second location may be adjusted in the first representation of at least the second location. In some approaches, the adjusted color is based on a safety parameter associated with the second location. For example, a first contour color may indicate a path that a robot takes to thereby show an area of the second location for the first user to avoid walking in order to avoid colliding with the robot. The adjusted color may additionally and/or alternatively indicate an area for the first user to safely be positioned in while at the second location.

Numerous benefits are enabled as a result of implementing the techniques of various embodiments and approaches described herein at a location such as an industrial floor. For example, as a result of performing one or more operations described herein, the level of difference in the illumination between a first and a second location are identified to seamlessly determine a representation for displaying on an MR device that will ultimately prevent a user from experiencing eye adaptation issues upon leaving the first location and/or entering the second location. This way, the user can perform seamless movement between dark to light surroundings and vice versa while corresponding representations are created and displayed to provide a MR surrounding for the user that is based on the user's rate of eye adaptation. It should also be noted that outputting representations, for display on a mixed reality (MR) device, having levels of light illumination that are based on a user's rate of eye adaptation has heretofore not been considered as a solution for eye adaptation issues commonly experienced in conventional settings. In sharp contrast, users themselves are forced to squint and pause their movement to adjust to differences in light at different locations. However, this is dangerous and inefficient. Accordingly, the inventive discoveries disclosed herein with regards to use of determining and outputting such representations proceed contrary to conventional wisdom.

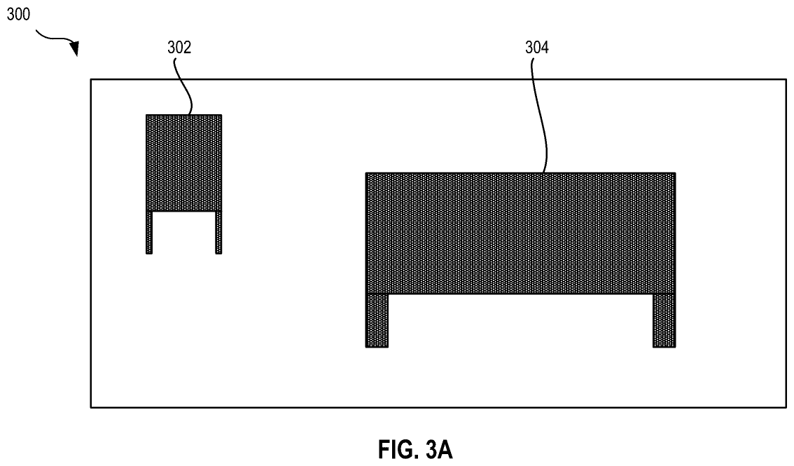

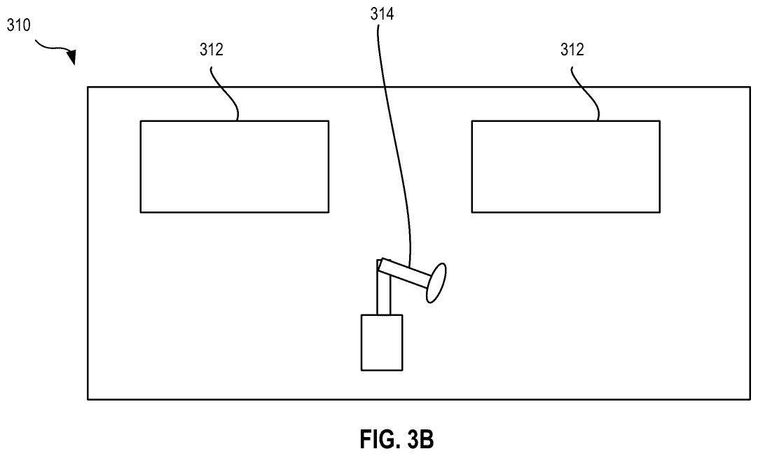

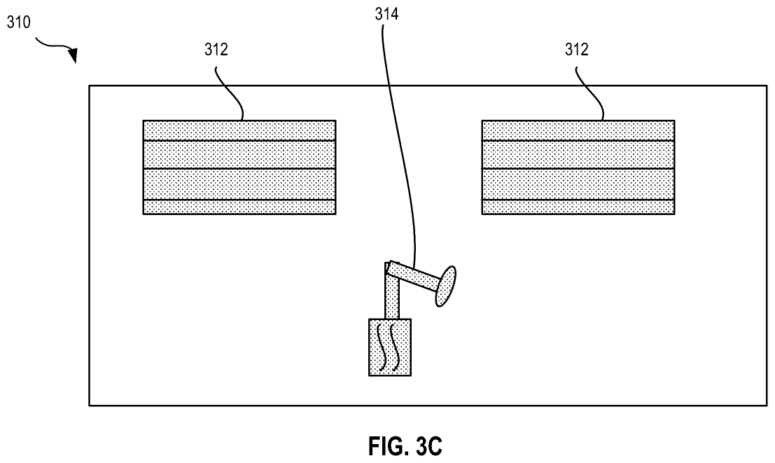

FIGS. 3A-3C depict perspectives 300, 310 of locations, in accordance with various embodiments. As an option, the present perspectives 300, 310 may be implemented in conjunction with features from any other embodiment listed herein, such as those described with reference to the other FIGS. Of course, however, such perspectives 300, 310 and others presented herein may be used in various applications and/or in permutations which may or may not be specifically described in the illustrative embodiments listed herein. Further, the perspectives 300, 310 presented herein may be used in any desired environment.

Referring first to FIG. 3A, the perspective 300 is a view that a user has in a relatively dark setting at a first location of an industrial floor. For example, the first location may be intentionally kept relatively dark based on photos being chemically developed. It may be noted that there is not a complete absence of light illumination at the first location because a plurality of objects, e.g., a chair 302 and a table 304, are somewhat visible in the perspective 300 with limited detail of the objects being able to be seen.

Referring now to FIG. 3B, the perspective 310 is a view that the user immediately has upon entering into a relatively light setting at a second location of an industrial floor. It may be assumed that the first location has a first level of light illumination, and a second location has a second level of light illumination that is relatively more than the first level, e.g., dark to light. Because of this, the user experiences eye adaptation issues upon entering into the second location. For example, it may be noted that two bookshelves 312 and a robotic arm 314 are visible the perspective 310 of the second location, but the user experiences a “white out” flash as the user's pupil muscle adjusts to the change in the perceived level of light illumination between the first location and the second location. This hurts the user's eyes and may potentially cause a headache for the user in accordance with experiencing the eye adaptation issues.

Referring now to FIG. 3C, it may be noted that the eye adaptation issues experienced by the user in the perspective 310 of FIG. 3B are not experienced by the user in the perspective 310 of FIG. 3C. This is because techniques described herein are implemented, e.g., such as those described in method 201. More specifically, perspective 310 of FIG. 3C may illustrate a display of a MR device that displays a first representation of the second location, where the first representation of the second location includes a level of light illumination that is determined to allow the user's pupil muscle to adjust at a learned rate of eye adaptation of the user. Accordingly, it may be noted that the contents of the second location are relatively clearer and more visible than those in the perspective 310 of FIG. 3B, and may be displayed until a determination is made that the user's eyes have adapted.

It will be clear that the various features of the foregoing systems and/or methodologies may be combined in any way, creating a plurality of combinations from the descriptions presented above.

It will be further appreciated that embodiments of the present invention may be provided in the form of a service deployed on behalf of a customer to offer service on demand.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, comprising:
learning a rate of eye adaptation that a first user's pupil muscle adjusts to a change in a perceived level of light illumination;
in response to a determination that the first user is moving from a first location having a first level of light illumination to a second location having a second level of light illumination, determining whether a difference in the levels of light illumination has a potential for causing eye adaptation issues for the first user;
in response to a determination that the difference in the levels of light illumination has a potential for causing eye adaptation issues for the first user, determining a level of light illumination that will allow the first user's pupil muscle to adjust at the learned rate of eye adaptation; and
outputting a first representation of the second location for display on a mixed reality (MR) device worn by the first user upon the first user entering the second location, wherein the first representation of the second location includes the determined level of light illumination.

2. The computer-implemented method of claim 1, wherein at least one of the locations is indoors, wherein the level of light illumination of the indoor location is output by a light emitting device.

3. The computer-implemented method of claim 2, wherein the levels of light illumination in the locations are not adjusted to allow the first user's pupil muscle to adjust at the learned rate of eye adaptation.

4. The computer-implemented method of claim 1, comprising:
receiving information associated with levels of light illumination of a plurality of other locations;
performing a process for each of the other locations, wherein the process includes:
determining whether a difference in the second level of light illumination and the level of light illumination of the other location has a potential for causing eye adaptation issues for the first user, and
in response to a determination that the difference in the second level of light illumination and the level of light illumination of the other location has a potential for causing eye adaptation issues for the first user, determining, a level of light illumination that will allow the first user's pupil muscle to adjust at the learned rate of eye adaptation;
storing the levels of light illumination to a predetermined table; and
referencing the predetermined table in response to a determination that the first user has left the second location and/or is headed to one of the other locations.

5. The computer-implemented method of claim 1, comprising:
updating, at a first predetermined rate, the level of light illumination of the first representation;
determining whether the first user's pupil muscle is adjusting at the learned rate of eye adaptation;
in response to a determination that the first user's pupil muscle is adjusting at about the learned rate of eye adaptation, continuing to update the level of light illumination of the first representation at the first predetermined rate; and
in response to a determination that the first user's pupil muscle is not adjusting at about the learned rate of eye adaptation, updating the level of light illumination of the first representation at a second predetermined rate.

6. The computer-implemented method of claim 1, comprising:
determining whether the first user's pupil muscle has fully adjusted to the second level of light illumination at the second location; and
in response to a determination that the first user's pupil muscle has fully adjusted to the second level of light illumination, performing a predetermined operation to cause the first user to view the second level of light illumination,
wherein the predetermined operation is selected from the group consisting of: not outputting anything to be displayed on the MR device worn by the first user, physically adjusting a display of the MR device so that the first user sees the second location without any portion of the MR device obstructing a view of the first user, and outputting a second representation of the second location for display on the MR device, wherein the second representation of the second location includes the second level of light illumination.

7. The computer-implemented method of claim 1, comprising: identifying a muscle flexion of the pupil of the first user, wherein the muscle flexion is considered during the learning of the rate of eye adaptation that the first user's pupil muscle adjusts.

8. The computer-implemented method of claim 7, wherein the learning is based on factors selected from the group consisting of: historical learning about the level of illumination, comfort level of one or more workers, and time.

9. The computer-implemented method of claim 1, comprising: adjusting a color of one or more articles of clothing of a second user in the first representation of the second location, wherein the adjusted color of the one or more articles of clothing is based on a job task of the second user.

10. The computer-implemented method of claim 1, comprising: adjusting a color of a contour of a floor of the second location in the first representation of at least the second location, wherein the adjusted color is based on a safety parameter associated with the second location.

11. A computer program product, the computer program product comprising a computer readable storage medium having program instructions embodied therewith, the program instructions readable and/or executable by a computer to cause the computer to:
learn, by the computer, a rate of eye adaptation that a first user's pupil muscle adjusts to a change in a perceived level of light illumination;
in response to a determination that the first user is moving from a first location having a first level of light illumination to a second location having a second level of light illumination, determine, by the computer, whether a difference in the levels of light illumination has a potential for causing eye adaptation issues for the first user;
in response to a determination that the difference in the levels of light illumination has a potential for causing eye adaptation issues for the first user, determine, by the computer, a level of light illumination that will allow the first user's pupil muscle to adjust at the learned rate of eye adaptation; and
output, by the computer, a first representation of the second location for display on a mixed reality (MR) device worn by the first user upon the first user entering the second location, wherein the first representation of the second location includes the determined level of light illumination.

12. The computer program product of claim 11, wherein at least one of the locations is indoors, wherein the level of light illumination of the indoor location is output by a light emitting device.

13. The computer program product of claim 12, wherein the levels of light illumination in the locations are not adjusted to allow the first user's pupil muscle to adjust at the learned rate of eye adaptation.

14. The computer program product of claim 11, the program instructions readable and/or executable by the computer to cause the computer to:
receive, by the computer, information associated with levels of light illumination of a plurality of other locations;
perform, by the computer, a process for each of the other locations, wherein the process includes:
determining whether a difference in the second level of light illumination and the level of light illumination of the other location has a potential for causing eye adaptation issues for the first user, and
in response to a determination that the difference in the second level of light illumination and the level of light illumination of the other location has a potential for causing eye adaptation issues for the first user, determining, a level of light illumination that will allow the first user's pupil muscle to adjust at the learned rate of eye adaptation;
store, by the computer, the levels of light illumination to a predetermined table; and
reference, by the computer, the predetermined table in response to a determination that the first user has left the second location and/or is headed to one of the other locations.

15. The computer program product of claim 11, the program instructions readable and/or executable by the computer to cause the computer to:
update, by the computer, at a first predetermined rate, the level of light illumination of the first representation;
determine, by the computer, whether the first user's pupil muscle is adjusting at the learned rate of eye adaptation;
in response to a determination that the first user's pupil muscle is adjusting at about the learned rate of eye adaptation, continue, by the computer, to update the level of light illumination of the first representation at the first predetermined rate; and
in response to a determination that the first user's pupil muscle is not adjusting at about the learned rate of eye adaptation, update, by the computer, the level of light illumination of the first representation at a second predetermined rate.

16. The computer program product of claim 11, the program instructions readable and/or executable by the computer to cause the computer to:
determine, by the computer, whether the first user's pupil muscle has fully adjusted to the second level of light illumination at the second location; and
in response to a determination that the first user's pupil muscle has fully adjusted to the second level of light illumination, perform, by the computer, a predetermined operation to cause the first user to view the second level of light illumination,
wherein the predetermined operation is selected from the group consisting of: not outputting anything to be displayed on the MR device worn by the first user, physically adjusting a display of the MR device so that the first user sees the second location without any portion of the MR device obstructing a view of the first user, and outputting a second representation of the second location for display on the MR device, wherein the second representation of the second location includes the second level of light illumination.

17. The computer program product of claim 11, the program instructions readable and/or executable by the computer to cause the computer to: identify, by the computer, a muscle flexion of the pupil of the first user, wherein the muscle flexion is considered during the learning of the rate of eye adaptation that the first user's pupil muscle adjusts.

18. The computer program product of claim 17, wherein the learning is based on factors selected from the group consisting of: historical learning about the level of illumination, comfort level of one or more workers, and time.

19. The computer program product of claim 11, the program instructions readable and/or executable by the computer to cause the computer to: adjust, by the computer, a color of one or more articles of clothing of a second user in the first representation of the second location, wherein the adjusted color of the one or more articles of clothing is based on a job task of the second user.

20. A system, comprising:
a processor; and
logic integrated with the processor, executable by the processor, or integrated with and executable by the processor, the logic being configured to:
learn a rate of eye adaptation that a first user's pupil muscle adjusts to a change in a perceived level of light illumination;
in response to a determination that the first user is moving from a first location having a first level of light illumination to a second location having a second level of light illumination, determine whether a difference in the levels of light illumination has a potential for causing eye adaptation issues for the first user;
in response to a determination that the difference in the levels of light illumination has a potential for causing eye adaptation issues for the first user, determine a level of light illumination that will allow the first user's pupil muscle to adjust at the learned rate of eye adaptation; and
output a first representation of the second location for display on a mixed reality (MR) device worn by the first user upon the first user entering the second location, wherein the first representation of the second location includes the determined level of light illumination.
